(19) United States
(12) Patent Application Publication
(10) Pub. No.: US 2017/0072141 A1
(43) Pub. Date: Mar. 16, 2017

(54) Syringe Assembly for Storing, Mixing, and Releasing Two Reactive Compounds

(71) Applicants: Warren Robinson, Elkins Park, PA (US); Chandricka Carr, Philadelphia, PA (US)

(72) Inventors: Warren Robinson, Elkins Park, PA (US); Chandricka Carr, Philadelphia, PA (US)

(21) Appl. No.: 15/342,559

(22) Filed: Nov. 3, 2016

Related U.S. Application Data
(63) Continuation-in-part of application No. 14/285,554, filed on May 22, 2014, now abandoned.
(60) Provisional application No. 61/826,386, filed on May 22, 2013.

(51) Int. Cl. A61M 5/315; A61M 5/31

(52) U.S. Cl. CPC A61M 5/31596; A61M 5/3129; A61M 5/31511; A61M 2005/3123; A61M 2005/3131; A61M 2202/064

(57) ABSTRACT

A syringe assembly that is pre-filled with two compounds. The two compounds are kept separate and are only mixed immediately before the compounds are ejected from the syringe. The syringe assembly has a syringe barrel. The syringe barrel defines an internal chamber where the two compound mix prior to ejection. Only one of the compounds is packaged within the syringe barrel. The other compound is introduced into the syringe barrel from a separate reservoir that is disposed within the syringe plunger. The second compound is released by twisting a reservoir tube within the syringe plunger.

### RELATED APPLICATIONS

This application is a continuation-in-part of co-pending patent application Ser. No. 14/285,554 filed May 22, 2014, which claims the benefit of provisional application Ser. No. 61/826,386, filed May 22, 2013.

### BACKGROUND OF THE INVENTION

1. Field Of The Invention

In general, the present invention relates to pre-filled syringe assemblies. More particularly, the present invention relates to syringe assemblies that hold two compounds in different compartments and enable those compounds to mix together just prior to dispensing from the syringe assembly.

2. Prior Art Description

Syringes are common used to inject compounds into the bodies of humans and animals. Syringes are also used to orally administer compounds. If the compound to be injected is a stable compound, then a syringe can be prefilled and stored for use. In fact, many prefilled syringes are produced and sold commercially by pharmacies. A problem occurs when the compound to be injected is a mixture that becomes unstable shortly after it is mixed. In such a scenario, the compound must be prepared and mixed by a knowledgeable professional, such as a pharmacist or other healthcare professional. The mixture must then be drawn into syringe and administered to a patient before the mixture has a chance to degrade.

In the prior art, there are many pharmaceutical compounds and nutraceutical compounds that are viable only for certain periods of time after mixing. Often, these compounds are a mixture of powered materials with a liquid solvent or a carrier fluid. These types of compounds are most often administered by health professionals who can mix the compounds just prior to their administration to a patient. However, such compounds are very difficult to commercialize in a manner that can be utilized directly by untrained users in the general public.

In the prior art, the problem has been addressed by developing double-chambered syringes. In a double-chambered syringe, a powdered compound can be kept in one chamber while a carrier liquid is kept isolated in a second chamber. Just prior to administration, the contents of the two chambers are mixed within the confines of the syringe. This is typically achieved by breaking or piercing a barrier within the syringe that separates the two chambers. Such prior art syringe assemblies are exemplified by U.S. Pat. No. 7,967,779 to Bertron, U.S. Pat. No. 7,329,235 to Bertron, U.S. Pat. No. 4,041,945 to Guiney, and U.S. Pat. No. 3,682,174 to Cohen.

A problem associated with such prior art double-chambered syringes is one of packaging integrity. Since a syringe is typically tubular, the two compounds are typically separated by a breakable barrier within a long tube structure. The membrane easily breaks as the plunger of the syringe is partially depressed. As a result, the system is packaged and sold with its plunger fully extended. Any pressure that is applied to the plunger through the packaging can move the plunger and damage its internal barrier. If an internal barrier is compromised, the compounds within can react and may become toxic over time. Accordingly, a new syringe that appears undamaged can easily become internally compromised and cause great harm to a patient.

A need therefore exists for a syringe that holds two isolated compounds that can be mixed prior to use, yet the syringe is designed to prevent mixing of the compounds even if its packaging is severely mishandled. In this manner, a safer syringe design is presented. This need is met by the present invention as described and claimed below.

### SUMMARY OF THE INVENTION

The present invention is a disposable syringe assembly that is pre-filled with two compounds. The two compounds are kept separate and are only mixed immediately before the compounds are ejected from the syringe. The syringe assembly has a syringe barrel. The syringe barrel defines an internal chamber where the two compound mix prior to ejection. Only one of the compounds is packaged within the syringe barrel. The other compound is introduced into the syringe barrel from a separate reservoir that is coupled to the syringe plunger.

The syringe plunger has a first end that extends into the syringe barrel. The syringe plunger defines an internal conduit and has perforations at one end. A reservoir tube is disposed within the internal conduit of the syringe plunger. The reservoir tube is selectively adjustable within the syringe plunger between a sealed position and an open position. When in the sealed position, the reservoir tube retains the second compound in the state of a liquid. When in the open position, the second compound is released into the internal conduit. The second compound flows through the perforations and into the internal chamber of the syringe barrel, therein mixing with the first compound. The syringe plunger is then advanced into the syringe barrel and the mixed compounds are ejected.

### BRIEF DESCRIPTION OF THE DRAWINGS

For a better understanding of the present invention, reference is made to the following description of an exemplary embodiment thereof, considered in conjunction with the accompanying drawings, in which:

FIG. 1 is a perspective view of an exemplary embodiment of a syringe assembly;

FIG. 2 is an exploded view of the exemplary embodiment of FIG. 1;

FIG. 3 is a cross-sectional view of the syringe assembly in an unused condition;

FIG. 4 is a cross-sectional view of the syringe assembly in a primed condition where compounds are mixed; and

FIG. 5 is a cross-sectional view of the syringe assembly in a condition ready for injection.

### DETAILED DESCRIPTION OF THE DRAWINGS

Although the present invention syringe assembly can be embodied in many ways, only one embodiment is presented for the purposes of illustration and discussion. This exemplary embodiment is selected in order to set forth one of the best modes contemplated for the invention. The illustrated embodiment, however, is merely exemplary and should not be considered a limitation when interpreting the scope of the appended claims.

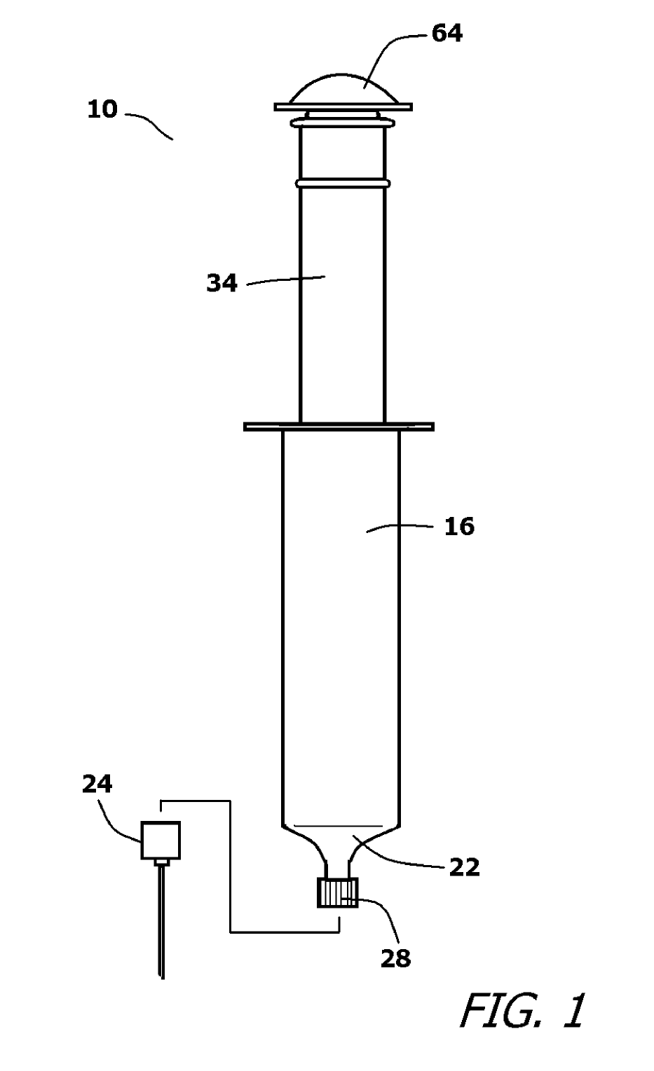

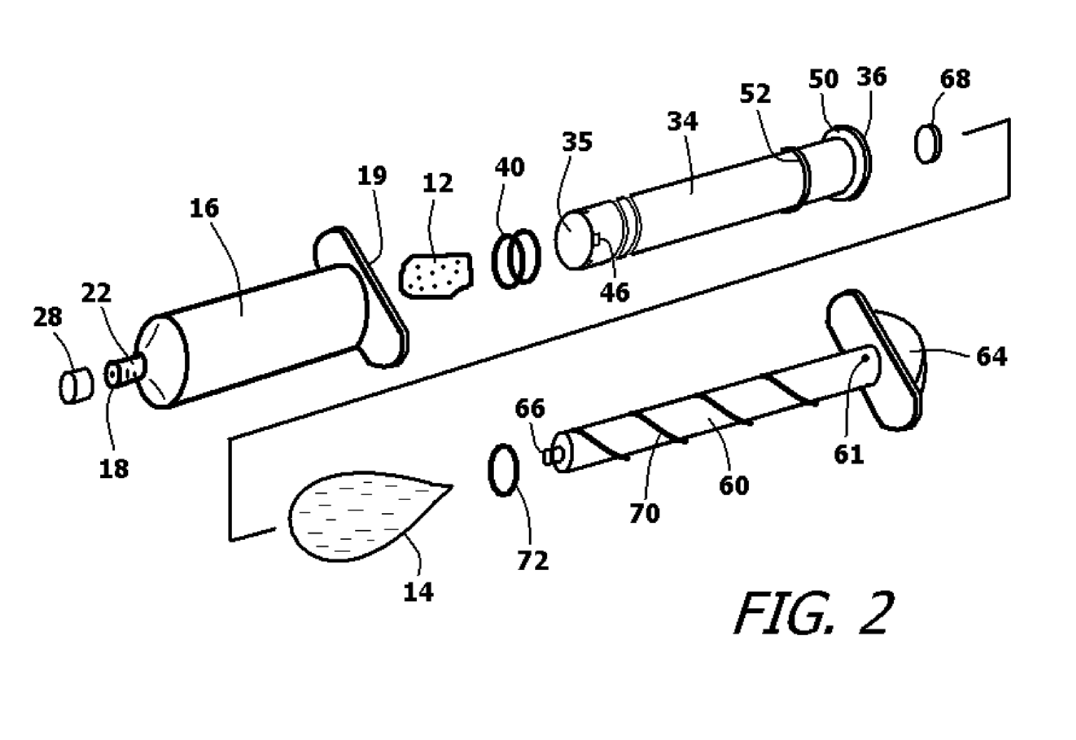

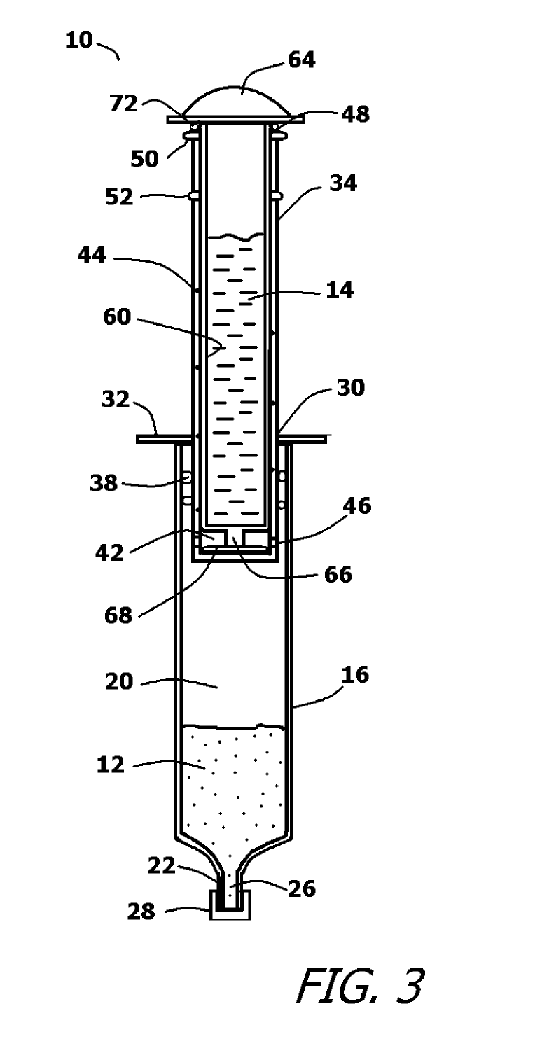

Referring to FIG. 1, FIG. 2 and FIG. 3, a disposable syringe assembly 10 is shown that contains a first compound 12 and a second compound 14. The two compounds 12, 14 are separately isolated within the structure of the syringe assembly 10. When the syringe assembly 10 is utilized, the first compound 12 and the second compound 14 are mixed together and can be ejected as a mixed compound from the syringe assembly 10.

The syringe assembly 10 has a primary barrel 16. The primary barrel 16 defines an internal dispensing chamber 20 between a first end 18 and a second end 19. The first end 18 of the primary barrel 16 is tapered to a narrow neck 22. The narrow neck 22 is shaped and sized to engage a cannula or other such syringe termination 24. Alternatively, the syringe assembly 10 can be used without a syringe termination for a direct oral administration of the compounds 12, 14. An opening 26 passes through the narrow neck 22 and communicates with the internal dispensing chamber 20. Prior to use, the opening 26 at the narrow neck 22 is sealed with a removable cap 28.

At the opposite second end 19 of the primary barrel 16 there is a large access opening 30 that leads into the internal dispensing chamber 20. A finger flange 32 radially extends from the primary barrel 16 proximate the large access opening 30. This finger flange 32 is engaged by a user's fingers when the syringe assembly 10 is manually compressed in the hand of a user.

A volume of the first compound 12 is set within the internal dispensing chamber 20 of the primary barrel 16. The first compound 12 can be either a liquid or a powder. The first compound 12 is prevented from exiting the internal dispensing chamber 20 through the narrow neck 22 by the removable cap 28. The first compound 12 is prevented from exiting the internal dispensing chamber 20 through the access opening 30 by the presence of a primary plunger 34.

The primary plunger 34 has a first end 35 and an opposite second end 36. A piston ring 38 is provided proximate the first end 35 of the primary plunger 34. The piston ring 38 can be an elastomeric head. However, in the shown embodiment, the piston ring 38 is made using at least one elastomeric O-ring 40. The O-ring 40 creates a liquid impervious seal between the exterior of the primary plunger 34 and the interior of the primary barrel 16.

The primary plunger 34 is tubular in structure and defines an internal conduit 42. A helical groove 44 is formed along the internal conduit 42 for a reason that is later explained. The first end 35 of the primary plunger 34 is perforated. The perforations 46 provide access to the internal conduit 42 through the first end 35. The second end 36 of the primary plunger 34 has a large opening 48. The large opening 48 provides access to the internal conduit 42 through the second end 36. An end flair 50 radially extends from the primary plunger 34 proximate its second end 36.

The primary plunger 34 is sized to pass into the dispensing chamber 20 of the primary barrel 16. The piston ring 38 creates a liquid impervious seal between the primary plunger 34 and the primary barrel 16. A stop 52 is formed on the exterior of the primary plunger 34 near the second end 36 of the primary plunger 34. The stop 52 is too large to pass into the primary barrel 16. The stop 52 is positioned so that it contacts the second end 19 of the primary barrel 16 at the same moment the primary plunger 34 fully displaces the first compound 12 from the primary barrel 16. In this manner, a user can know that the primary barrel 16 has been fully emptied by advancing the primary plunger 34 into the primary barrel 16 until the stop 52 on the primary plunger 34 contacts the primary barrel 16 and prevents any further advancement of the primary plunger 34.

A reservoir tube 60 is provided. The reservoir tube 60 is connected to a knob head 64 at one end. The reservoir tube 60 holds a volume of the second compound 14. The second compound 14 is in liquid form. The second compound 14 can exit the reservoir tube 60 through a flow port 66 at the end of the reservoir tube 60 opposite the knob head 64. To prevent a vapor lock, a small vent hole 61 is formed in the reservoir tube 60 near the knob head 64. Prior to use, the flow port 66 is closed by being pressed against a seal gasket 68. The seal gasket 68 is impervious to the second compound 14 and prevents any of the second compound 14 from inadvertently exiting the reservoir tube 60.

A helical thread 70 is formed on the exterior of the reservoir tube 60. The helical thread 70 engages the helical groove 44 on the interior of the primary plunger 34. Accordingly, the reservoir tube 60 threads onto the interior of the primary plunger 34. The reservoir tube 60 can therefore be advanced and retracted into and out of the primary plunger 34 by turning the reservoir tube 60 relative the primary plunger 34.

The reservoir tube 60 is turned by manually engaging the knob head 64 at the distal end of the reservoir tube 60. To help inhibit the knob head 64 from inadvertently turning, an O-ring 72 is placed around the reservoir tube 60 under the knob head 64. The O-ring 72 is compressed between the end flare 50 of the primary plunger 34 and the knob head 64. As such, the resiliency of the O-ring 72 resists the turning of the knob head 64. The O-ring 72 also creates an airtight seal which prevents air from reaching the vent hole 61 in the reservoir tube 60.

Referring to FIG. 3 in conjunction with FIG. 2, it can be seen that in its packaged, unused condition (shown in FIG. 3), the reservoir tube 60 is tightened into the primary plunger 34. In this position, the O-ring 72 between the knob head 64 of the reservoir tube 60 and the end flair 50 of the primary plunger 34 is compressed. Furthermore, the flow port 66 at the end of the reservoir tube 60 presses against the seal gasket 68. This prevents any of the second compound 14 in the reservoir tube 60 from escaping. As a result, the first compound 12 in the primary barrel 16 is kept separate and distinct from the second compound 14 in the reservoir tube 60.

If the syringe assembly 10 is accidentally compressed during shipping or storage, the primary plunger 34 may be advanced further into the primary barrel 16. However, this causes no harm. The movement of the primary plunger 34 has no affect on the release of the second compound 14 in the reservoir tube 60. Since the dispensing first end 18 of the primary barrel 16 is sealed with a cap 28, the first compound 12 cannot be displaced. As such, inadvertent compression of the syringe assembly 10 does not harm the syringe assembly 10 or compromise the compounds 12, 14 stored within the syringe assembly 10 in any way.

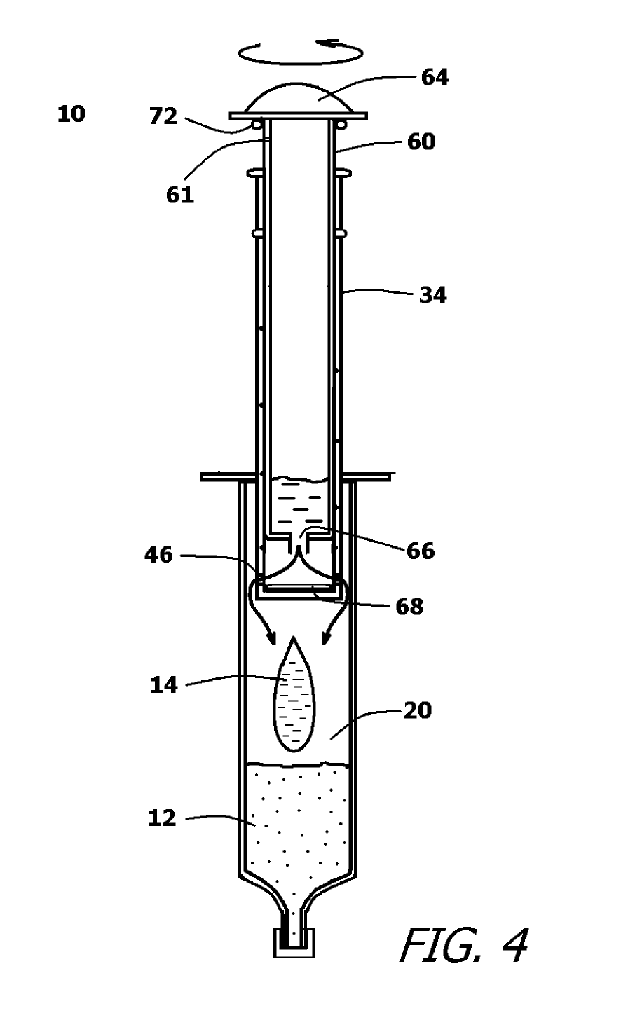

Referring now to FIG. 4 in conjunction with FIG. 2, it can be seen that in order to activate the syringe assembly 10, the knob head 64 is turned to raise the reservoir tube 60 out of the primary plunger 34. This does two things. First, it breaks the seal of the O-ring 72 under the knob head 64. This enables air to reach the vent hole 61 on the reservoir tube 60. Second, the raising of the reservoir tube 60 pulls the flow port 66 of the reservoir tube 60 away from the seal gasket 68. This unblocks the flow port 66 and enables the second compound 14 to flow out through the flow port 66.

Once the flow port 66 of the reservoir tube 60 opens, the second compound 14 flows into the interior of the primary plunger 34 near its first end 35. The first end 35 of the primary plunger 34 is perforated. As such, the secondary compound 14 flows through the perforations 46 and into the dispensing chamber 20 of the primary barrel 16. Within the dispensing chamber 20, the primary compound 12 mixes with the secondary compound 14.

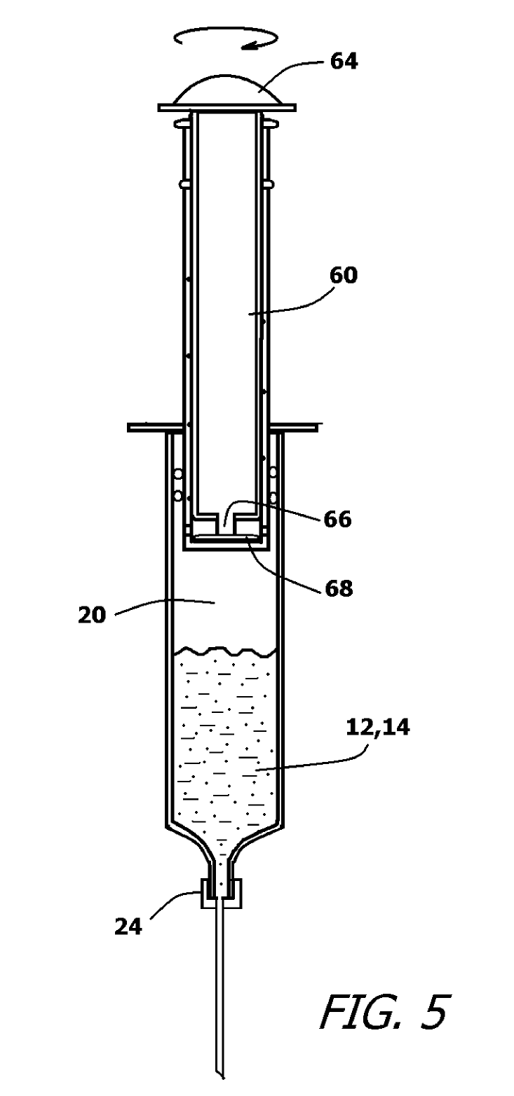

Referring lastly to FIG. 5, it can be seen that the knob head 64 is again rotated to seat the flow port 66 of the reservoir tube 60 against the seal gasket 68. This prevents any material from reentering the reservoir tube 60. The syringe assembly 10 can then be shaken to mix the first compound 12 with the second compound 14 in the dispensing chamber 20. The cap 28 is removed from the neck 22 of the primary barrel 16 and a cannula or other syringe termination 24 is attached. Alternatively, the syringe assembly 10 can be used directly to orally administer the mixture of the first compound 12 and a second compound 14. The reservoir tube 60 and the primary plunger 34 are the advanced in unison into the primary barrel 16. This displaces the mixture of the first compound 12 and the second compound 14 out of the syringe assembly 10.

It will be understood that the embodiment of the present invention that is illustrated and described is merely exemplary and that a person skilled in the art can make many variations to that embodiment. For instance, the shape of the turn knob, the volume of the reservoir tube and the volume of the primary barrel are all a matter of design choice. All such embodiments are intended to be included within the scope of the present invention as defined by the claims.

What is claimed is:

1. A syringe assembly comprising:
a syringe barrel having a dispensing end and an opposite open end, wherein said syringe barrel defines an internal chamber between said open end and said dispensing end;
a compound disposed within said internal chamber;
a syringe plunger having a first end that extends into said syringe barrel through said open end of said syringe barrel, wherein said syringe plunger defines an internal conduit that is accessible through at least one hole in said syringe plunger proximate said first end of said syringe plunger;
a reservoir tube disposed within said internal conduit of said syringe plunger, wherein said reservoir tube is selectively adjustable within said syringe plunger between a sealed position and an open position, wherein when in said sealed position said reservoir tube retains a liquid, and wherein when in said open position said liquid is released into said internal conduit, wherein said liquid flows through said at least one hole into said internal chamber of said syringe barrel, therein mixing said liquid with said compound.

2. The assembly according to claim 1, wherein said compound is a powered material.

3. The assembly according to claim 1, further including a knob head that is coupled to said reservoir tube, therein enabling said reservoir tube to be selectively turned by manual manipulation between said sealed position and said open position.

4. The assembly according to claim 1, further including a seal gasket disposed within said internal conduit of said syringe plunger, wherein said reservoir tube is pressed against said seal gasket in said sealed position and is separated from said seal gasket in said open position.

5. The assembly according to claim 1, wherein said reservoir tube engages said internal conduit of said syringe plunger with a threaded connection, wherein said reservoir tube moves between said sealed position and said open position by turning said reservoir tube within said threaded connection.

6. The syringe assembly according to claim 1, further including a vent hole in said reservoir tube, wherein said vent hole is sealed when said reservoir tube is in said sealed position and is open when said reservoir tube is in said open position.

7. The syringe assembly according to claim 1, wherein said syringe plunger and said reservoir tube are movable as a unit into said internal chamber of said syringe barrel to displace said compound from said syringe barrel.

8. A syringe assembly comprising:
a syringe barrel that defines an internal chamber;
a first compound disposed within said internal chamber;
a syringe plunger that extends into said syringe barrel, wherein said syringe plunger defines an internal conduit;
a reservoir tube disposed within said internal conduit of said syringe plunger, wherein said reservoir tube retains a second compound and is manually adjustable within said syringe plunger between a first position and a second position, wherein when in said first position said reservoir tube retains said second compound, and wherein when in said second position, said second compound is released into said internal chamber of said syringe barrel, therein mixing said first compound with said second compound.

9. The assembly according to claim 8, wherein said first compound is a powered material.

10. The assembly according to claim 9, wherein said second compound is a liquid.

11. The assembly according to claim 8, further including a knob coupled to said reservoir tube, therein enabling said reservoir tube to be selectively turned between said first position and said second position.

12. The assembly according to claim 8, further including a seal gasket disposed within said internal conduit of said syringe plunger, wherein said reservoir tube is pressed against said seal gasket in said first position and is separated from said seal gasket in said second position.

13. The assembly according to claim 8, wherein said reservoir tube engages said internal conduit of said syringe plunger with a threaded connection, wherein said reservoir tube moves between said first position and said second position by turning said reservoir tube within said threaded connection.

14. The syringe assembly according to claim 8, further including a vent hole in said reservoir tube, wherein said vent hole is sealed when said reservoir tube is in said first position and is open when said reservoir tube is in said second position.

15. The syringe assembly according to claim 8, wherein said syringe plunger and said reservoir tube are both advanced into said internal chamber of said syringe barrel to displace said first compound from said syringe barrel.

16. A syringe assembly comprising:
a syringe barrel that defines an internal chamber;
a first compound disposed within said internal chamber;
a syringe plunger that extends into said syringe barrel;
a reservoir tube that extends into said syringe plunger, wherein said reservoir tube is selectively rotatable within said syringe plunger between a first position and a second position, wherein when in said first position, said reservoir tube retains a second compound, and wherein when in said second position, said second compound is released into said syringe barrel, therein mixing said first compound with said second compound.

17. The assembly according to claim 16, wherein said first compound is a powered material.

18. The assembly according to claim 17, wherein said second compound is a liquid.

19. The assembly according to claim 16, further including a knob coupled to said reservoir tube, for selectively turning said reservoir tube between said first position and said second position.

20. The assembly according to claim 16, further including a seal gasket disposed within said syringe plunger, wherein said reservoir tube is pressed against said seal gasket in said first position and is separated from said seal gasket in said second position.
